(19) United States
(12) Patent Application Publication
(10) Pub. No.: US 2013/0267825 A1
(43) Pub. Date: Oct. 10, 2013

(54) METHODS FOR DIAGNOSIS AND MONITORING OF NEUROLOGIC DISEASES USING MAGNETIC RESONANCE METHODS

(71) Applicant: MOLECULAR IMAGE, INC., Redwood City, CA (US)

(72) Inventor: Jay WOHLGEMUTH, Menlo Park, CA (US)

(21) Appl. No.: 13/627,994

(22) Filed: Sep. 26, 2012

Related U.S. Application Data
(63) Continuation of application No. 12/518,272, filed on Jun. 8, 2009, now abandoned.
(60) Provisional application No. 60/873,791, filed on Dec. 8, 2006.

(51) Int. Cl. A61B 5/055

(52) U.S. Cl. CPC A61B 5/055
USPC 600/410

(57) ABSTRACT

A method of diagnosing or monitoring a neurological condition in a subject is described. The method includes performing a first magnetic resonance method on a subject to produce a first data set, performing a second magnetic resonance method on the subject to produce a second data set, and analyzing the first data set and the second data set.

### CROSS REFERENCE TO RELATED APPLICATIONS

The present application is a continuation of U.S. application Ser. No. 12/518,272, filed Dec. 7, 2007, which is a national stage of PCT/US07/86837, filed Dec. 7, 2007, which claims priority to U.S. Provisional Application No. 60/873,791, filed Dec. 8, 2006, which are all incorporated herein by reference in their entirety.

### BACKGROUND

Disorders of the brain are serious medical conditions causing disability and diminished quality of life. Neurological damage is largely irreversible and thus early diagnosis and close monitoring are critical to the successful treatment of patients. Brain tissue is not readily available for histological evaluation or other diagnostic procedures due to the morbidity associated with brain biopsy. Medical imaging including magnetic resonance imaging is a mainstay of diagnosis and monitoring of neurologic diseases.

Multiple sclerosis (MS) is an autoimmune and inflammatory disease of the central nervous system characterized by unpredictable episode of brain inflammation and damage. An estimated 400,000 Americans are known to have MS. MS is one of the most common neurological diseases affecting young adults. The onset of symptoms usually occurs between the ages of 20 and 40 years old effecting young women and men in the prime of their lives. Conventional MRI is used very frequently to diagnose and monitor MS, but detects disease only after significant damage is done and thus does not completely enable prevention of irreversible neurological damage associated with disease. Efficacious therapies are available for MS, but are expensive and have significant toxicities and side effects. Thus, it is not appropriate to treat all patients with MS with these therapies in the absence of evidence of ongoing of impending disease activity. Early initiation of therapy in patients at risk reduces progression of MS (Kappos et al. Neurology, 2006; 67: 1242-1249, Jacobs et al. NEJM, 2000; 343: 898-904). A means to identify patients who are at either high or low risk for MS disease activity or to monitor therapy could prevent neurologic complications and reduce the costs and complications of unnecessary treatment. Current diagnostics and imaging techniques are inadequately sensitive and predictive of disease activity and neurologic complications and thus there is a significant need for improved or novel approaches.

Alzheimer's disease (AD) is a neurodegenerative disease associated with progressive memory loss and cognitive dysfunction. An estimated 4 million Americans have AD. By the year 2030 approximately 1 in every 80 persons in the U.S. will have AD.

From the time of diagnosis, people with AD survive about half as long as those of similar age without dementia. Medicare costs for beneficiaries with AD were $91 billion in 2005 and may increase to as much as $160 billion in 2010. Finding a treatment that could delay the onset by five years could reduce the number of individuals with AD by nearly 50 percent after 50 years. Drug development for AD is very active and sensitive imaging technologies could identify patients for therapy and monitor their response. Improved sensitivity of imaging tools for AD would thus be a significant boon to drug development for this disease and would also provide a means to guide therapeutic decision making thus improving outcomes and reducing unnecessary exposure of patients to costly medications with unwanted side effects.

Magnetic resonance imaging (MRI) and magnetic resonance spectroscopy (MRS) have both been applied to patients with MS and AD. MRI based techniques (including MRS) offer advantages over many other imaging approaches due to excellent anatomical resolution, lack of patient exposure to ionizing radiation and availability in most major hospitals. MRI is routinely used for MS and AD patient monitoring, but is not adequately sensitive or predictive of neurological outcomes (for example see Brex et al. 2002).

In MS, MRI provides information on the anatomical findings and identifies lesions in the brain due to MS, but once it is able to detect these lesions, irreversible damage to the central nervous system has already been done. Conventional MRI can be used to assess the presence, location and extent of brain lesions on T1 or T2 weighted MRI (Polman et al. 2006, Miller et al. 2003 Arnold et al. 2002, Brex et al. 2002). MRI can also be used to measure brain volume or volume of a particular brain structure or region as brain tissue tends to shrink due to the effects of MS disease activity on neurons and myelin sheaths (Arnold et al. 2002). MRI can also be used to detect the disruption of white matter axonal tracts using diffusion tensor MRI, a technique with some demonstrated value in monitoring MS disease activity and progression (Goldberg-Zimring et al, 2006, Hesseltine et al. 2006, Vrenken et al. 2006, Ge et al. 2005, Goldberg-Zimring et al. 2005). Magnetization transfer imaging with MRI takes advantage of water associated hydrogens to detect changes in normal appearing white matter and gray matter of patients with MS (Sharma et al. 2006, Agosta et al. 2006, Oreja-Guevara et al. 2006, Rocca et al. 2004, Filippi et al, 2004). Individually, these techniques all provide some value in monitoring MS patients however they fail to provide adequate predictive value for patient outcomes and would be much more powerful if used in combination with each other and MRS techniques (Brex et al. 2002, Narayana et al. 2005).

In AD, MRI scanning can be used to rule out other potential causes of dementia or can be used to measure the volume of the brain or the volume of specific brain structures (e.g., hippocampus, entorhinal cortex) which are known to shrink with AD progression (see Dickerson et al. 2005). However, individual MRI methods are not adequately sensitive, specific or predictive to enable optimal management of patients with AD.

Magnetic resonance spectroscopy (MRS) is a method by which MRI systems can be used to measure peaks associated with specific metabolites in tissues in vivo. The technique takes advantage of specific resonances from protons or other atoms which are unique to specific molecular entities. These techniques can be used to measure the levels of several metabolites which are of known importance and relevance to brain chemistry, function and inflammation and to disease of the brain such as MS and AD. For general reviews on MRS concepts and their application to MS and AD, see Narayana et al. 2005, Dickerson et al. 2005, Kantarci et al. 2004, Gonzalez-Toledo et al. 2006, Lin et al. 2005.

MRS techniques have been shown to provide valuable information for diagnosis and management of patients with neurological diseases which is not available using conventional MRI (Narayana et al. 2005, Lin et al. 2005, Gonzalez-Toledo et al. 2006). MRI techniques have also been shown to have value as well. However, there are a number of shortcomings of existing methods which have impeded their clinical use and have limited the value of the information they provide. Chief among these is the fact that existing methods are not standardized or automated which results in increased variability in measurements and decreased value of the information. In addition, methods are currently used individually which would be more sensitive, predictive and/or reproducible if used in combination with additional complimentary methods.

### BRIEF SUMMARY

Described herein are methods of diagnosing or monitoring a neurological condition in a subject comprising: (a) performing a first magnetic resonance method on said subject to produce a first data set, (b) performing a second magnetic resonance method on said subject to produce a second data set, and (c) analyzing said first data set and second data set to diagnose or monitor a neurological condition or disease in said subject.

In some variations, the first and second data sets are selected from the group consisting of images, levels of a metabolite and measurements of a magnetic resonance property of a tissue. In some variations, the first data set is compared to an atlas. In some variations, the second data set is compared to an atlas.

In some variations, the first magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, volumetric measurements of brain, brain structures or lesions, T1 weighted MRI, T2 weighted MRI, T1 weighted MRI with contrast agents, and T2 weighted MRI with contrast agents.

In some variations, the brain structure is selected from the group consisting of corpus callosum, parietal lobes-posterior horns, hippocampus, and entorhinal cortex of hippocampus. In some variations, the anatomical magnetic resonance imaging uses volumetric measurements of whole brain, lesions, or specific brain structures.

In some variations, the second magnetic resonance method is magnetic resonance spectroscopy (MRS). In some variations, the magnetic resonance spectroscopy (MRS) is a single voxel method or a multi voxel method. In some variations, the multi-voxel method is chemical shift imaging.

In some variations, MRS includes analyzing the amount of one or more metabolites. In some variations, one or more metabolites are selected from the group consisting of myoinositol, NAA, choline, lipids, lactate, N-acetylaspartate glutamate, glutamine and creatine. In some variations, the magnetic resonance spectroscopy measures levels of one or more metabolites selected from the group consisting of lipids, lactate, N-acetylaspartate, glutamate, glutamine, creatine, choline, and myo-inositol. In some variations, the magnetic resonance spectroscopy measures A/B wherein A is the amount of a first metabolite and B is the amount of a second metabolite, wherein said first metabolite and second metaboilite are selected from the group consisting of free lipids, fatty acid species, lactate, N-acetylaspartate, glutamate, glutamine, choline, and myo-inositol. In some variations, the magnetic resonance spectroscopy measures the ratio of C/D wherein C is the sum of the amounts of two or more metabolites and D is the sum of two or more metabolites, wherein C/D does not equal one and wherein the metabolites are selected from the group consisting of free lipids, fatty acid species, lactate, N-acetylaspartate, glutamate, glutamine, choline, and myo-inositol.

In some variations, the first magnetic resonance method is anatomical magnetic resonance imaging and said second magnetic resonance method is magnetic resonance spectroscopy.

In some variations, the first magnetic resonance method is diffusion tensor imaging and said second magnetic resonance method is magnetic resonance spectroscopy.

In some variations, the first magnetic resonance method is magnetization transfer imaging and said second magnetic resonance method is magnetic resonance spectroscopy.

In some variations, the imaging methods are diffusion tensor imaging and magnetization transfer imaging.

In some variations, the methods further comprise administering a contrast agent to said subject. In some variations, the contrast agent is selected from the group consisting of gadolinium, gadodiamide, gadopentetate dimeglumine, gadoteridol, and gadoterate meglumine.

In some variations, the neurological condition diagnosed or monitored affects the brain or spinal cord. In some variations, the neurological condition is multiple sclerosis. In some variations, the neurological condition is Alzheimer's disease.

Described herein are methods of diagnosing or monitoring a neurological disease or condition in a subject, comprising: (a) performing a first magnetic resonance method on said subject to produce a first data set; (b) performing a second magnetic resonance method on said subject to produce a second data set; (c) repeating step (a), or repeating step (b) or repeating step (a) and step (b) to generate additional data sets; and (d) analyze said first data set, said second data set and said additional data sets to diagnose or monitor a neurological disease or condition in said subject.

In some variations, steps (a)-(d) are performed before and after treatment with a drug or therapy. In some variations step (a) and step (b) occur prior to treatment of said neurological disease or condition with a drug or therapy and step (c) occurs after treatment with said drug or therapy. In some variations, step (a) and step (b) occur during treatment of said neurological disease or condition with a drug or therapy and step (c) occurs after said treatment.

In some variations, the first data set is calculated as a change per unit volume of the brain, brain region, brain structure, brain lesion of spinal cord region, structure or lesion. In some variations, the second data set is calculated as a change per unit volume of the brain, brain region, brain structure, brain lesion of spinal cord region, structure or lesion.

In some variations, the first data set is compared to an atlas. In some variations, the second data set is compared to an atlas. In some variations, the additional data sets are compared or registered to an atlas.

In some variations, the first and second data sets are selected from the group consisting of images, levels of a metabolite and measurements of a magnetic resonance property of a tissue.

In some variations, the first magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, volumetric measurements of brain structures or lesions, T1 weighted MRI, T2 weighted MRI, T1 weighted MRI with contrast agents, and T2 weighted MRI with contrast agents.

In some variations, the brain structure is selected from the group consisting of corpus callosum, parietal lobes-posterior horns, hippocampus, and entorhinal cortex of hippocampus. In some variations, the anatomical magnetic resonance imaging uses volumetric measurements of whole brain, lesions, or specific brain structures.

In some variations, the second magnetic resonance method is magnetic resonance spectroscopy (MRS). In some variations, the magnetic resonance spectroscopy (MRS) is a single voxel method or a multi voxel method. In some variations, the multi-voxel method is chemical shift imaging.

In some variations, MRS includes analyzing one or more metabolites. In some variations, one or more metabolites are selected from the group consisting of myoinositol, NAA, choline, lipids, lactate, N-acetylaspartate glutamate, glutamine and creatine. In some variations, the magnetic resonance spectroscopy measures levels of one or more metabolites selected from the group consisting of lipids, lactate, N-acetylaspartate, glutamate, glutamine, creatine, choline, and myo-inositol. In some variations, the magnetic resonance spectroscopy measures A/B wherein A is the amount of a first metabolite and B is the amount of a second metabolite, wherein said first metabolite and second metaboilite are selected from the group consisting of free lipids, fatty acid species, lactate, N-acetylaspartate, glutamate, glutamine, choline, and myo-inositol. In some variations, the magnetic resonance spectroscopy measures the ratio of C/D wherein C is the sum of the amounts of two or more metabolites and D is the sum of two or more metabolites, wherein C/D does not equal one and wherein the metabolites are selected from the group consisting of free lipids, fatty acid species, lactate, N-acetylaspartate, glutamate, glutamine, choline, and myo-inositol.

In some variations, the methods further comprise administering a contrast agent to said subject. In some variations, the contrast agent is selected from the group consisting of gadolinium, gadodiamide, gadopentetate dimeglumine, gadoteridol, and gadoterate meglumine.

In some variations, the neurological condition diagnosed or monitored affects the brain or spinal cord. In some variations, the neurological condition is multiple sclerosis. In some variations, the neurological condition is Alzheimer's disease.

Described herein are methods of diagnosing or monitoring a neurological condition in a subject, comprising: (a) identifying a brain region, a brain lesion or a brain structure with a first magnetic resonance method; (b) performing a second magnetic resonance method on said brain region, brain lesion or brain structure to produce a data set and; (c) analyzing said data set to diagnose or monitor a neurological condition in said subject.

In some variations, step (a) further includes measuring the volume of said brain region, brain lesion or brain structure. In some variations, the methods further include performing a volume correction on said data set. In some variations, the data set is compared to an atlas. In some variations, the data set is selected from the group consisting of images, levels of a metabolite and measurements of a magnetic resonance property of a tissue.

In some variations, the first magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, volumetric measurements of brain structures or lesions, T1 weighted MRI, T2 weighted MRI, T1 weighted MRI with contrast agents, and T2 weighted MRI with contrast agents.

In some variations, the brain structure is selected from the group consisting of corpus callosum, parietal lobes-posterior horns, hippocampus, and entorhinal cortex of hippocampus. In some variations, the anatomical magnetic resonance imaging uses volumetric measurements of whole brain, lesions, or specific brain structures.

In some variations, the second magnetic resonance method is magnetic resonance spectroscopy (MRS). In some variations, the magnetic resonance spectroscopy (MRS) is a single voxel method or a multi voxel method. In some variations, the multi-voxel method is chemical shift imaging.

In some variations, MRS includes analyzing one or more metabolites. In some variations, one or more metabolites are selected from the group consisting of myoinositol, NAA, choline, lipids, lactate, N-acetylaspartate glutamate, glutamine and creatine. In some variations, the magnetic resonance spectroscopy measures levels of one or more metabolites selected from the group consisting of lipids, lactate, N-acetylaspartate, glutamate, glutamine, creatine, choline, and myo-inositol. In some variations, the magnetic resonance spectroscopy measures A/B wherein A is the amount of a first metabolite and B is the amount of a second metabolite, wherein said first metabolite and second metaboilite are selected from the group consisting of free lipids, fatty acid species, lactate, N-acetylaspartate, glutamate, glutamine, choline, and myo-inositol. In some variations, the magnetic resonance spectroscopy measures the ratio of C/D wherein C is the sum of the amounts of two or more metabolites and D is the sum of two or more metabolites, wherein C/D does not equal one and wherein the metabolites are selected from the group consisting of free lipids, fatty acid species, lactate, N-acetylaspartate, glutamate, glutamine, choline, and myo-inositol.

In some variations, the methods further comprise administering a contrast agent to said subject. In some variations, the contrast agent is selected from the group consisting of gadolinium, gadodiamide, gadopentetate dimeglumine, gadoteridol, and gadoterate meglumine.

In some variations, the neurological condition diagnosed or monitored affects the brain or spinal cord. In some variations, the neurological condition is multiple sclerosis. In some variations, the neurological condition is Alzheimer's disease.

Described herein are methods of diagnosing or monitoring a neurological disease or condition in a subject, comprising; (a) performing a first magnetic resonance method on said subject to produce a first data set; (b) performing a second magnetic resonance method on said subject to produce a second data set; (c) performing a third magnetic resonance method on said subject to produce a third data set and; (d) analyzing said first data set, said second data set, and said third data set to diagnose or monitor a neurological condition or disease in said subject.

In some variations, the first data set is compared to an atlas. In some variations, the second data set is compared to an atlas. In some variations, the third data set is compared to an atlas.

In some variations, the first, second, and third data sets are selected from the group consisting of images, levels of a metabolite and measurements of a magnetic resonance property of a tissue.

In some variations, the first magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, volumetric measurements of brain, brain structures or lesions, T1 weighted MRI, T2 weighted MRI, T1 weighted MRI with contrast agents, and T2 weighted MRI with contrast agents.

In some variations, the brain structure is selected from the group consisting of corpus callosum, parietal lobes-posterior horns, hippocampus, and entorhinal cortex of hippocampus. In some variations, the anatomical magnetic resonance imaging uses volumetric measurements of whole brain, lesions, or specific brain structures.

In some variations, the second magnetic resonance method is magnetic resonance spectroscopy (MRS). In some variations, the magnetic resonance spectroscopy (MRS) is a single voxel method or a multi voxel method. In some variations, the multi-voxel method is chemical shift imaging.

In some variations, MRS includes analyzing one or more metabolites. In some variations, one or more metabolites are selected from the group consisting of myoinositol, NAA, choline, lipids, lactate, N-acetylaspartate glutamate, glutamine and creatine. In some variations, the magnetic resonance spectroscopy measures levels of one or more metabolites selected from the group consisting of lipids, lactate, N-acetylaspartate, glutamate, glutamine, creatine, choline, and myo-inositol. In some variations, the magnetic resonance spectroscopy measures A/B wherein A is the amount of a first metabolite and B is the amount of a second metabolite, wherein said first metabolite and second metaboilite are selected from the group consisting of free lipids, fatty acid species, lactate, N-acetylaspartate, glutamate, glutamine, choline, and myo-inositol. In some variations, the magnetic resonance spectroscopy measures the ratio of C/D wherein C is the sum of the amounts of two or more metabolites and D is the sum of two or more metabolites, wherein C/D does not equal one and wherein the metabolites are selected from the group consisting of free lipids, fatty acid species, lactate, N-acetylaspartate, glutamate, glutamine, choline, and myo-inositol.

In some variations, the methods further comprise administering a contrast agent to said subject. In some variations, the contrast agent is selected from the group consisting of gadolinium, gadodiamide, gadopentetate dimeglumine, gadoteridol, and gadoterate meglumine.

In some variations, the neurological condition diagnosed or monitored affects the brain or spinal cord. In some variations, the neurological condition is multiple sclerosis. In some variations, the neurological condition is Alzheimer's disease.

Described herein are methods of diagnosing or monitoring a neurological condition in a subject, comprising: (a) measuring the volume of a brain, a brain region, a brain lesion or a brain structure with a first magnetic resonance method to produce a first data set; (b) performing a second magnetic resonance method on said brain, brain region, brain lesion or brain structure to produce a second data set and; (c) analyzing said first and second data set to diagnose or monitor a neurological condition in said subject.

In some variations, the methods further include performing a volume correction on said data set. In some variations, the first data set is compared to an atlas. In some variations, the second data set is compared to an atlas.

In some variations, the second data sets are selected from the group consisting of images, levels of a metabolite and measurements of a magnetic resonance property of a tissue.

In some variations, the first magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, T1 weighted MRI, T2 weighted MRI, T1 weighted MRI with contrast agents, and T2 weighted MRI with contrast agents.

In some variations, the brain structure is selected from the group consisting of corpus callosum, parietal lobes-posterior horns, hippocampus, and entorhinal cortex of hippocampus.

In some variations, the second magnetic resonance method is magnetic resonance spectroscopy (MRS). In some variations, the magnetic resonance spectroscopy (MRS) is a single voxel method or a multi voxel method. In some variations, the multi-voxel method is chemical shift imaging.

In some variations, MRS includes analyzing one or more metabolites. In some variations, one or more metabolites are selected from the group consisting of myoinositol, NAA, choline, lipids, lactate, N-acetylaspartate glutamate, glutamine and creatine. In some variations, the magnetic resonance spectroscopy measures levels of one or more metabolites selected from the group consisting of lipids, lactate, N-acetylaspartate, glutamate, glutamine, creatine, choline, and myo-inositol. In some variations, the magnetic resonance spectroscopy measures A/B wherein A is the amount of a first metabolite and B is the amount of a second metabolite, wherein said first metabolite and second metaboilite are selected from the group consisting of free lipids, fatty acid species, lactate, N-acetylaspartate, glutamate, glutamine, choline, and myo-inositol. In some variations, the magnetic resonance spectroscopy measures the ratio of C/D wherein C is the sum of the amounts of two or more metabolites and D is the sum of two or more metabolites, wherein C/D does not equal one and wherein the metabolites are selected from the group consisting of free lipids, fatty acid species, lactate, N-acetylaspartate, glutamate, glutamine, choline, and myo-inositol.

In some variations, the methods further comprise administering a contrast agent to said subject. In some variations, the contrast agent is selected from the group consisting of gadolinium, gadodiamide, gadopentetate dimeglumine, gadoteridol, and gadoterate meglumine.

In some variations, the neurological condition diagnosed or monitored affects the brain or spinal cord. In some variations, the neurological condition is multiple sclerosis. In some variations, the neurological condition is Alzheimer's disease.

Described herein are computer-readable storage mediums containing compute-executable instructions for diagnosis or monitoring a neurological disease or condition in a subject comprising instructions to: (a) obtain a first data set from said subject wherein the first data set is produced using a first magnetic resonance method; (b) obtain a second data set from said subject wherein the second data set is produced using a second magnetic resonance method and; (c) analyze said first and second data sets to diagnose or monitor a neurological condition or disease in said subject.

Described herein are computer-readable storage mediums containing compute-executable instructions for diagnosis or monitoring a neurological disease or condition in a subject comprising instructions to: (a) obtain a first data set from said subject wherein the first data set is produced using a first magnetic resonance method; (b) obtain a second data set from said subject wherein the second data set is produced using a second magnetic resonance method; (c) repeat step (a), step (b) or step (a) and step (b) to generate additional data sets and; (d) analyze said first data set, said second data set and said additional data sets to diagnose or monitor a neurological disease or condition in said subject.

Described herein are computer-readable storage mediums containing compute-executable instructions for diagnosis or monitoring a neurological disease or condition in a subject comprising instructions to: (a) identify a brain region, a brain lesion or a brain structure with a first magnetic resonance method; (b) obtain a second data set on said brain region, brain lesion or brain structure wherein the second data set is produced using a second magnetic resonance method, and; (c) analyze said data set to diagnose or monitor a neurological condition in said subject.

Described herein are computer-readable storage mediums containing compute-executable instructions for diagnosis or monitoring a neurological disease or condition in a subject comprising instructions to: (a) obtain a first data set from said subject wherein the first data set is produced using a first magnetic resonance method; (b) obtain a second data set from said subject wherein the second data set is produced using a second magnetic resonance method; (c) obtain a third data set from said subject wherein the third data set is produced using a third magnetic resonance method and; (d) analyze said first data set, said second data set, and said third data set to diagnose or monitor a neurological condition or disease in said subject.

Described herein are computer-readable storage mediums containing compute-executable instructions for diagnosis or monitoring a neurological disease or condition in a subject comprising instructions to: (a) obtain a first data set from said subject wherein the first data set measures the volume of a brain, a brain region, a brain lesion or a brain structure using a first magnetic resonance method; (b) obtain a second data set from said subject wherein the second data set is produced using a second magnetic resonance method and; (c) analyze said first and second data set to diagnose or monitor a neurological condition in said subject.

Described herein are methods of diagnosing or monitoring a neurological condition in a subject comprising: (a) performing a magnetic resonance method on said subject to determine a volume; (b) performing magnetic resonance spectroscopy on said subject to produce a first data set, wherein said first data set comprises the value of the ratio C/D wherein C is the amount of a first metabolite and D is the amount of a second metabolite wherein C/D is not equal to 1, wherein said first metabolite and second metabolite are selected from the group consisting of free lipids, fatty acid species, lactate, N-acetylaspartate, glutamate, glutamine, choline, and myo-inositol, and (c) analyzing said volume and first data set to diagnose or monitor a neurological condition or disease in said subject.

In some variations, the magnetic resonance method is magnetic resonance imaging (MRI). In some variations, the magnetic resonance imaging (MRI) method is selected from the group consisting of diffusion tensor imaging (DTI), anatomical resonance imaging, magnetization transfer imaging, volumetric measurements of brain, brain structures or lesions, T1 weighted MRI, T2 weighted MRI, T1 weighted MRI with contrast agents, and T2 weighted MRI with contrast agents.

In some variations, the magnetic resonance spectroscopy (MRS) is a single voxel method or a multi voxel method.

### DETAILED DESCRIPTION

The present methods pertain to using two or more imaging methods or techniques in combination for the diagnosis and/or monitoring of neurological conditions and/or diseases. The result is improvement in reproducibility, sensitivity, specificity and/or predictive value of the methods and thus improved management of patients with neurological diseases such as AD and MS. The methods pertain to two or more methods which result in improved performance when used together. The methods are also a method for diagnosis and monitoring of patients with neurologic diseases including MS and AD. The methods are also a software package or kit which implements two or more methods or techniques for imaging of patients with neurologic diseases where the combined method has improved performance relative to either method alone.

### Magnetic Resonance Imaging (MRI)

Magnetic resonance imaging (MRI) is a noninvasive medical imaging technique that uses the interaction between radio frequency pulses, a strong magnetic field and body tissue to obtain images of slices/planes from inside the body. These magnets generate fields from approximately 2000 times up to 30000 times stronger than that of the Earth. The use of magnetic resonance principles produces extremely detailed pictures of the body tissue without the need for x-ray exposure and gives diagnostic information of various organs.

Measured are mobile hydrogen nuclei (protons are the hydrogen atoms of water, the ‘H’ in H20), the majority of elements in the body. Only a small part of them contribute to the measured signal, caused by their different alignment in the magnetic field. Protons are capable of absorbing energy if exposed to short radio wave pulses (electromagnetic energy) at their resonance frequency. After the absorption of this energy, the nuclei release this energy so that they return to their initial state of equilibrium. This transmission of energy by the nuclei as they return to their initial state is what is observed as the MRI signal. The subtle differing characteristic of that signal from different tissues combined with complex mathematical formulas analyzed on modern computers is what enables MRI imaging to distinguish between various organs. Any imaging plane, or slice, can be projected, and then stored or printed.

The measured signal intensity depends jointly on the spin density and the relaxation times (T1 time and T2 time), with their relative importance depending on the particular imaging technique and choice of interpulse times. Any motion such as blood flow, respiration, etc. also affects the image brightness.

Magnetic resonance imaging is particularly sensitive in assessing anatomical structures, organs and soft tissues for the detection and diagnosis of a broad range of pathological conditions. MRI pictures can provide contrast between benign and pathological tissues and may be used to stage cancers as well as to evaluate the response to treatment of malignancies. The need for biopsy or exploratory surgery can be eliminated in some cases, and can result in earlier diagnosis of many diseases (See Huk W. J. and Gademann G., (1984) Magnetic resonance imaging (MRI): method and early clinical experiences in diseases of the central nervous system Neurosurg Rev. 7(4):259-80).

### Diffusion Tensor Imaging

Diffusion Tensor Imaging (DTI) (also referred to as diffusion tensor MRI) is the measure of tensor directly from diffusion-weighted data. A tensor is used to describe diffusion in anisotropic systems. Diffusion tensor imaging is the more sophisticated form of diffusion weighted imaging, which allows for the determination of directionality as well as the magnitude of water diffusion. The fractional anisotropy (FA) gives information about the shape of the diffusion tensor at each voxel. The FA is based on the normalized variance of the given values.

The fractional anisotropy reflects differences between an isotropic diffusion and a linear diffusion. The FA range is between 0 and 1 (0=isotropic diffusion, 1=highly directional) (See Jones, D. (2005) Fundamentals of Diffusion MR Imaging, CLINICAL MR NEUROIMAGING DIFFUSION, PERFUSION AND SPECTROSCOPY, Gillard J. et al., Cambridge, Cambridge Univ. Press: 54-85).

DTI allows the visualization of the location, orientation and anisotropy of the brain's white matter tracts. White matter diffusion property preferentially orients in one direction called anisotropic diffusion. Applying diffusion gradients in diffusion MRI, in at least 6 directions, it is possible to calculate a tensor (i.e. a 3×3 matrix) that describes the 3-dimensional shape of diffusion. The fiber direction will be indicated by the tensor's main eigenvector. DTI is useful in studying tractography (the orientation of white matter tracts in fibers within the brain) within white matter.

### Magnetization Transfer Imaging

Magnetization Transfer Imaging (MTI) (also referred to as Magnetization Transfer MRI) is based on the magnetization interaction (through dipolar and/or chemical exchange) between bulk water protons and macromolecular protons (See Grossman R. I. et al. (1994) Magnetization transfer: theory and clinical applications in neuroradiology Radiographics 14:279-290). By applying an off resonance radio frequency pulse to the macromolecular protons, the saturation of these protons is then transferred to the bulk water protons. The result is a decrease in signal (the net magnetization of visible protons is reduced), depending on the magnitude of MT between tissue macromolecules and bulk water. With MTI, the presence or absence of macromolecules (e.g. in membranes, brain tissue) can be seen. Magnetization transfer techniques make demyelinated brain or spine lesions (as seen e.g. in multiple sclerosis) better visible on T2 weighted images as well as on gadolinium contrast enhanced T1 weighted images.

Another type of magnetization transfer imaging is magnetization transfer contrast (MTC). MTC increases the contrast by removing a portion of the total signal in tissue. An off resonance radio frequency (RF) pulse saturates macromolecular protons to make them invisible (caused by their ultra-short T2* relaxation times) (See Wolff S. D. and Balaban R. S. (1989) Magnetization transfer contrast (MTC) and tissue water proton relaxation in vivo Magn Reson Med. 10(1):135-44). The signal from semi-solid tissue like brain parenchyma is reduced, and the signal from a more fluid component like blood is retained.

Off resonance makes use of a selection gradient during an off resonance MTC pulse. The gradient has a negative offset frequency on the arterial side of the imaging volume (caudally more off resonant and cranially less off resonant). The net effect of this type of pulse is that the arterial blood outside the imaging volume will retain more of its longitudinal magnetization, with more vascular signal when it enters the imaging volume. Off resonance MTC saturates the venous blood, leaving the arterial blood untouched.

On resonance has no effect on the free water pool but will saturate the bound water pool and is the difference in T2 between the pools. Special binomial pulses are transmitted causing the magnetization of the free protons to remain unchanged. The z-magnetization returns to its original value. The spins of the bound pool with a short T2 experience decay, resulting in a destroyed magnetization after the on resonance pulse.

### Magnetization Resonance Spectroscopy

The underlying principle of Magnetization Resonance Spectroscopy (MRS) is that atomic nuclei are surrounded by a cloud of electrons, which very slightly shield the nucleus from any external magnetic field. As the structure of the electron cloud is specific to an individual molecule or compound, then the magnitude of this screening effect is also a characteristic of the chemical environment of individual nuclei (See Danielsen E. and Ross B. D. (1999) Magnetic resonance spectroscopy diagnosis of neurological diseases, New York Marcel-Dekker; see also Lin, A. et al. (2005) Efficacy of proton magnetic resonance spectroscopy in neuorological diagnosis and neurotherapeutic decision making NeuroRx 2(2): 197-214).

In view of the fact that the resonant frequency is proportional to the magnetic field that it experiences, it follows that the resonant frequency will be determined not only by the external applied field, but also by the small field shift generated by the electron cloud. This shift in frequency is called the chemical shift. The chemical shift is a very small effect, usually expressed in ppm of the main frequency. In order to resolve the different chemical species, it is therefore necessary to achieve very high levels of homogeneity of the main magnetic field B0. Spectra from humans usually require shimming the magnet to approximately one part in 100. High resolution spectra of liquid samples demand a homogeneity of about one part in 1000.

In addition to the effects of factors such as relaxation times that can affect the NMR signal, as seen in magnetic resonance imaging, effects such as J-modulation or the transfer of magnetization after selective excitation of particular spectral lines can affect the relative strengths of spectral lines.

In the context of human MRS, two nuclei are of particular interest—H-1 and P-31. (PMRS—Proton Magnetic Resonance Spectroscopy) PMRS is mainly employed in studies of the brain where prominent peaks arise from NAA, choline containing compounds, creatine and creatine phosphate, myo-inositol, glutamate and glutamine, and, if present, lactate; phosphorus 31 MR spectroscopy detects compounds involved in energy metabolism (creatine phosphate, adenosine triphosphate and inorganic phosphate) and certain compounds related to membrane synthesis and degradation. n-Acetyl aspartate (NAA) is a marker of healthy neurons and axons and low or decreasing levels of this marker measured by MRS are associated with neuronal loss. In MS and AD measurement of this marker by MRS in the brain has been shown to have some utility in diagnosis and monitoring of patients (For example see Narayana et al. 2005, Lin et al. 2005, Gonen et al. 2002, Adalsteinsson et al. 2000, Ross et al. U.S. Pat. No. 5,617,861, Arnold et al. U.S. Pat. No. 6,347,239, Pfefferbaum et al. U.S. Pat. No. 6,819,952). Myo-inositol is thought to be a marker of glial cell proliferation associated with brain inflammation and has also been measured with MRS in patients with AD and MS (Vrenken et al. 2005, Fernando et al. 2004, Pfefferbaum et al. U.S. Pat. No. 6,819,952). Free lipids measured by MRS may be increased with damage to myelin sheaths which occurs as a critical part of the MS disease process (Narayana et al. 2005). Choline is felt to be a marker of demyelination and may also be of use in MS or other diseases associated with loss of myelin (Lin et al. 2005). Glutamate and glutamine are bioamines used as excitatory neurotransmitters in the brain and have been found to be elevated in MS brain tissue and MS brain lesions using MRS (Srinivasan et al. 2005). Measurement of these metabolites may provide insight into the molecular events of neurological disease processes which may be sensitive for early disease, predictive of future events and more sensitive and predictive than conventional MRI, other imaging techniques or other diagnostic tests.

If the field is uniform over the volume of the sample, “similar” nuclei will contribute a particular frequency component to the detected response signal irrespective of their individual positions in the sample. Since nuclei of different elements resonate at different frequencies, each element in the sample contributes a different frequency component. A chemical analysis can then be conducted by analyzing the MR response signal into its frequency components.

The frequencies of certain lines may also be affected by factors such as the local pH. It is also possible to determine intracellular pH because the inorganic phosphate peak position is pH sensitive.

H1 (proton) MRS may be used or MRS for other nuclei. MRS data may be acquired with a short echo time (such as TE 35 ms) or any other echo time. Acquisition of a scout image may be a part of the method as well as standardized selection of MRS slices for multi-voxel approaches and single voxel locations. Mutli-voxel chemical shift imaging may be used as well as single voxel methods. Single voxel methods or analysis of data from multi-voxel methods may be obtained from standardized regions within the white or gray matter of the brain such as from the corpus callosum the parietal lobes (e.g., posterior horns), or other standard peri-ventricular white matter area or other standard gray matter area. Areas of interest may also include the posterior cingulate gyrus, hippocampus or entorhinal cortex of the hippocampus. For references on these MRS methods see Narayana et al. 2005, Dickerson et al. 2005, Kantarci et al. 2004, Gonzalez-Toledo et al. 2006, Lin et al. 2005, Gonen et al. 2002, Adalsteinsson et al. 2000, Ross et al. U.S. Pat. No. 5,617,861, Arnold et al. U.S. Pat. No. 6,347,239, Pfefferbaum et al. U.S. Pat. No. 6,819,952, Vrenken et al. 2005, Fernando et al. 2004, Srinivasan et al. 2005.

MRS data may be calculated for the entire brain and may be corrected for volume of the whole brain. MRS data may also be calculated for standard regions of the brain described above and may be volume corrected for these regions, structures of lesions. Data may be compared to a previous scan of the same patient with or without therapy being administered in the interim. This may involve a method of registration of the image and data to prior scan using a variety of techniques and calculation of change metrics for all parameters or metabolites in all regions. Rate of change can also be calculated which includes consideration of the time interval between serial scans. Metabolites can be measured by MRS in the entire brain or in specific anatomical structures or locations seen on MRI. For example, metabolites can be measured in the white matter, the gray matter or in lesions in MS patients. They can be measured in the hippocampus or the posterior cingulate gyrus in AD. Levels of these metabolites can normalized to creatine levels in a tissue which is a constitutive marker. They can also be measured serially in patients over time in the same location with or without intervening therapy to determine the change or rate of change in the brain or a brain region. Alternatively they may be measured in relationship to one another which may provide a meaningful metric for the disease process.

### Combining Imaging Methods

Combining multiple imaging approaches for evaluation of neurological diseases can improve the information value from the scan including an increase in sensitivity or specificity. In addition, the combination of multiple methods in an algorithm or protocol can lead to improved reproducibility (decreased variability) of the data derived from these methods. Using ratios of MRS peaks, volume correction of MRS data and registration of image from serial scans or registration of a scan to an atlas can all improve the quality of the data and reduce variability in the measurements. Use of methods in combination thus improves both the value and relevance of the information and reduces variability of each measurement which results in increased clinical utility for patients. In order to implement these combined imaging techniques in a highly reproducible manner, a standardized protocol is developed and software to implement this method is developed. The software plays a key role in processing and combining data from multiple modalities and controlling and standardizing the data processing, quality control and analysis procedures which results in decreased variability of the methods. A multi-center validation study of the combined method is then performed to prove the reproducibility and clinical value for a specific disease state.

Most importantly, MRI and MRS methods have increased value when multiple, complimentary methods are used in combination for evaluation of a patient. For example, information on metabolites in the brain obtained from MRS can be combined with anatomical MRI information to enhance the value of the information. For example, MRS data may be obtained from the brain or from a brain region, but that brain region may shrink in size over time due to the disease process. Therefore, it may be appropriate to correct MRS data for the brain area or the area of the brain region from which it's measured. Another example is the use of a brain atlas or image registration method to ensure that MRS data is measured from the correct anatomical location in the brain and so that serial examinations with MRS can be compared from the same anatomical location. Further, MRS data from the brain can be combined in algorithms which also include findings from anatomical MRI scanning such as the number or volume of lesions seen with T1 or T2 weighted imaging or the results of diffusion tensor imaging or the results of magnetization transfer imaging approaches.

The methods pertain to using more than one imaging method or technique in combination in order to improve on the clinical value of the information. The methods of diagnosing or monitoring a neurological condition in a subject may comprise performing a first magnetic resonance method on the subject and performing a second magnetic resonance method to diagnose or monitor a neurological condition or disease in the subject. The first and second magnetic resonance methods may include (MRI), diffusion tensor imaging (DTI), diffusion weighted imaging (DWI), anatomical resonance imaging, magnetization transfer imaging, magnetization transfer contrast, volumetric measurements of brain, brain structures or lesions, T1 weighted MRI, T2 weighted MRI, T1 weighted MRI with contrast agents, T2 weighted MRI with contrast agents, and/or MRS.

The methods pertain to the use of any two or more of the magnetic resonance methods in combination when combining the methods results in some improvement in reproducibility, sensitivity, specificity, predictive power or ease of use for clinicians. Combinations can be of 2 or more, 3 or more, 4 or more, etc. methods. Multiple methods may be combined in the method of the invention including those methods referenced herein as well as additional methods.

The methods of diagnosing or monitoring a neurological condition in a subject may comprise identifying a brain region, a brain lesion or a brain structure with a first magnetic resonance method and performing a second magnetic resonance method to diagnose or monitor a neurological condition in the subject.

The methods of diagnosing or monitoring a neurological condition in a subject may comprising measuring the volume of a brain, a brain region, a brain lesion or a brain structure with a first magnetic resonance method and performing a second magnetic resonance method on the brain, brain region, brain lesion or brain structure to diagnose or monitor a neurological condition in the subject.

The methods of diagnosing or monitoring a neurological disease or condition in a subject may comprise (a) performing a first magnetic resonance method, (b) performing a second magnetic resonance method on the subject, and (c) repeating step (a), or repeating step (b) or repeating step (a) and step (b) to generate additional data sets to diagnose or monitor a neurological disease or condition in the subject. The steps may be performed before and/or after treatment with a drug or therapy. Steps (a) and (b) may occur prior to treatment of said neurological disease or condition with a drug or therapy and step (c) may occur after treatment with said drug or therapy. Steps (a) and (b) may occur during treatment of said neurological disease or condition with a drug or therapy and step (c) may occur after said treatment.

In the context of the described methods, an imaging method may be image registration to previous images or to a brain or spinal cord atlas, volumetric measurements of whole brain, lesions or specific brain structures using anatomical MRI, serial measurements of volumes, image data processing algorithms, quality control processes such as assessment of signal to noise, line width criteria, and location of voxels slices. Methods also include magnetic resonance spectroscopy for one metabolite or multiple metabolites or ratios or other combinations of metabolites. Metabolites measured by MRS may include but are not limited to free lipids, fatty acid species, myo-inositol, n-acytyl aspartate (NAA), choline, creatine, glutamate and glutamine. Methods also include mapping metabolites measured by MRS to anatomical MRI images. Methods also include measuring metabolites with MRS using single voxel or multi-voxel approaches (e.g., chemical shift imaging).

The methods of diagnosing or monitoring a neurological condition in a subject may comprise performing a magnetic resonance method and performing magnetic resonance spectroscopy, wherein the magnetic resonance spectroscopy is used to determine the value of the ratio of amount of a metabolite or multiple metabolites to diagnose or monitor a neurological condition or disease in the subject. In some variations the metabolites may be free lipids, fatty acid species, lactate, N-acetylaspartate, glutamate, glutamine, choline, and myo-inositol.

The combined imaging method is validated in a multi-center clinical trial in which the method is standardized across multiple clinical centers and imaging systems using a software package. Patient with a target disease indication are enrolled and the method is implemented and shown to have value in diagnosis, risk stratification, prediction of disease course or outcomes, selection of therapy, monitoring of therapy or monitoring for complications of disease or therapy (for examples of patient groups and study endpoints see for example Miller et al. 2003, Narayana et al. 2005, Gonzalez-Toledo et al. 2006, Lin et al. 2005, Kantarci et a;. 2004, Fernando et al. 2004, Gonen et al. 2002). Endpoints in the studies may include current of future clinical manifestations of disease (e.g., dementia or disability) or current or future findings on MRI or other imaging methods (e.g., brain atrophy or shrinkage or brain lesions).

Individual methods may include methods of data acquisition which may be performed on all available clinical MRI imaging systems (e.g., Phillips, GE and Siemens). Data may be acquired using 1.5T and 3T clinical systems or greater field strengths (see Kantarci et al. 2003). Methods may include the use of anatomical MRI of the whole brain or of specific brain regions or brain lesions (Polman et al. 2006, Miller et al. 2003 Arnold et al. 2002, Brex et al. 2002). Diffusion tensor imaging may be used (Goldberg-Zimring et al, 2006, Hesseltine et al. 2006, Vrenken et al. 2006, Ge et al. 2005, Goldberg-Zimring et al. 2005) as well as magnetization transfer imaging (Sharma et al. 2006, Agosta et al. 2006, Oreja-Guevara et al. 2006, Rocca et al. 2004, Filippi et al, 2004).

Raw data may be transferred from the clinical imaging system to a processing server and reading of data may be performed with a variety of techniques including reading of data on server using LC model or JMRUI or other approaches (Vanhamme et al. 1997, van den Boogaart et al. 1996, Kapeller et al. 2005, Hancu et al. 2005).

Data processing and analysis may include image unwarping, atlas based alignment and segmentation steps. Identification of standard brain regions for analysis may be performed using atlas based registration methods (for example Dale et al. 2002 US 2003/013959). Quality control steps may be included which take into account signal to noise measures in specific locations, line width criteria and the anatomical location of single voxels or multi-voxel slices. Anatomical MRI calculations may include calculation of whole brain area, calculation of area of specific structures, regional cortical thickness, calculation of individual and overall lesion area, diffusion tensor imaging results (fractional anisotropy and mean diffusivity) for standard locations, magnetization transfer results for standard locations, and MT ratio quantification within suregional white matter and gray matter. MRI data may be T1 or T2 weighted and may be with or without the use of contrast. MRS calculations may include calculation of peaks and ratios for any number of metabolites including but not limited to myoinositol, NAA, Choline, Cr, free lipids, Glutamine/Glutamate. For references on these MRS methods see Narayana et al. 2005, Dickerson et al. 2005, Kantarci et al. 2004, Gonzalez-Toledo et al. 2006, Lin et al. 2005, Gonen et al. 2002, Adalsteinsson et al. 2000, Ross et al. U.S. Pat. No. 5,617,861, Arnold et al. U.S. Pat. No. 6,347,239, Pfefferbaum et al. U.S. Pat. No. 6,819,952, Vrenken et al. 2005, Fernando et al. 2004, Srinivasan et al. 2005.

The method may include data display and reporting including QC information, brain areas, structure areas, segmental volumes, regional cortical thickness, lesion load, longitudinal change, with error bounds and reference ranges, anatomical MRI results with heat maps shown for diffusion tensor imaging results, magnetization transfer and results for MRS. Reporting may include metabolite values for the whole brain or standard regions and lesions with reference ranges and error bounds. Reporting may also be for volume corrected or longitudinal results. Reporting may be numerical, graphical or heatmaps of results superimposed on anatomical MRI images. Reporting metrics may be for any measurement individually or for any combination of parameters. Reporting may also provide some interpretation of the results including a diagnosis or comparison to a reference population.

### Contrast Agents

The invention also provides for use of contrast agents in the methods of the invention. Contrast agents are chemical substances introduced to the anatomical or functional region being imaged, to increase the differences between different tissues or between normal and abnormal tissue, by altering the relaxation times. Contrast agents are classified by the different changes in relaxation times after their injection.

Positive contrast agents cause a reduction in the T1 relaxation time (increased signal intensity on T1 weighted images). They (appearing bright on MRI) are typically small molecular weight compounds containing as their active element Gadolinium, Manganese, or Iron. All of these elements have unpaired electron spins in their outer shells and long relaxivities. Some typical contrast agents as gadopentetate dimeglumine, gadoteridol, and gadoterate meglumine are utilized for the central nervous system and the complete body; mangafodipir trisodium is specially used for lesions of the liver and gadodiamide for the central nervous system.

Negative contrast agents (appearing predominantly dark on MRI) are small particulate aggregates often termed superparamagnetic iron oxide (SPIO). These agents produce predominantly spin relaxation effects (local field inhomogeneities), which results in shorter T1 and T2 relaxation times. SPIO's and ultrasmall superparamagnetic iron oxides (USPIO) usually consist of a crystalline iron oxide core containing thousands of iron atoms and a shell of polymer, dextran, polyethyleneglycol, and produce very high T2 relaxivities. USPIOs smaller than 300 nm cause a substantial T1 relaxation. T2 weighted effects are predominant. A special group of negative contrast agents (appearing dark on MRI) are perfluorocarbons (perfluorochemicals), because their presence excludes the hydrogen atoms responsible for the signal in MR imaging.

(Gd) Gadolinium is a Lanthanide element that is paramagnetic in its trivalent state. This paramagnetic substance is used for MR imaging because of the effect of strongly decreasing the T1 relaxation times of the tissues to which gadolinium has access. When injected during magnetic resonance imaging, gadolinium will tend to change signal intensities by shortening the T1 time in its surroundings. The gadolinium ion cannot be used in its chloride, sulfate, or acetate forms because of poor tolerance and low solubility in water in the neutral pH range. Although toxic by itself, gadolinium can be given safely in a chelated form such as DTPA that still retains much of its strong effect on relaxation times.

Macromolecular paramagnetic contrast agents are being tested worldwide. Preclinical data shows that these agents demonstrate great promise for improving the quality of MR angiography, and in quantificating capillary permeability and myocardial perfusion. Further, ultrasmall superparamagnetic iron oxide (USPIO) particles have been evaluated in multicenter clinical trials for lymph node MR imaging and MR angiography, with the clinical impact under discussion. In addition, a wide variety of vector and carrier molecules, including antibodies, peptides, proteins, polysaccharides, liposomes, and cells have been developed to deliver magnetic labels to specific sites.

### Software Package and Kit

One embodiment of the invention is a software package which implements each step in the method including data acquisition from the imaging system, processing of raw data, quality control, calculation of results including combining results from multiple methods, reporting and display of results and provision of an interpretation. In some cases, this software package takes the form of a kit which is implemented by a user of the technique.

Diagnosis and/or Monitoring of Neurological Conditions

For the purposes of this description the term “diagnosis” or “diagnosis and monitoring” is used to encompass numerous clinical uses of the invention for management of patients with neurological diseases. For example, the invention may be used to diagnose the presence of disease for the first time, to risk stratify patients with a diagnosis of disease into higher and lower risk groups, prediction of disease activity, flares or clinical progression, predicting response to a therapy prior to administration of a drug or after administration of a drug, selection of a specific therapy, selecting a patient for a clinical trial of a new therapy, or using the invention as an endpoint in a clinical trial of a therapeutic.

The invention can be applied to any neurological condition. Specifically the invention is useful for patient with possible or confirmed MS or AD, optic neuritis, clinically isolated syndrome, dementia of unknown cause.

The neurological condition may be a neurological disease including, but not limited, to multiple sclerosis. Multiple sclerosis (abbreviated MS, also known as disseminated sclerosis) is a chronic, inflammatory disease that affects the central nervous system (CNS). Multiple sclerosis affects neurons, the cells of the brain and spinal cord that carry information, create thought and perception, and allow the brain to control the body. Surrounding and protecting some of these neurons is a fatty layer known as the myelin sheath, which helps neurons carry electrical signals. MS causes gradual destruction of myelin (demyelination) and transection of neuron axons in patches throughout the brain and spinal cord. This scarring causes symptoms which vary widely depending upon which signals are interrupted. It is thought that MS results from attacks by an individual's immune system on the nervous system and is therefore categorized as an autoimmune disease. MS primarily affects adults, with an age of onset typically between 20 and 40 years, and is more common in women than in men (Calabresi P. A., (2004) Diagnosis and management of multiple sclerosis Am Fam Physician 70(10): 1935-44).

The neurological condition may be a neurological disease including, but not limited, to Alzheimer's disease. Alzheimer's disease (AD) has been identified as a protein misfolding disease due to the accumulation of abnormally folded amyloid beta protein in the brains of AD patients (Hashimoto M et al. (2003) Role of protein aggregation in mitochondrial dysfunction and neurodegeneration in Alzheimer's and Parkinson's diseases Neuromolecular Med 4 (1-2): 21-36). Although amyloid beta monomers are soluble and harmless, they undergo a dramatic conformational change at sufficiently high concentration to form a beta sheet-rich tertiary structure that aggregates to form amyloid fibrils that deposit outside neurons in dense formations known as senile plaques or neuritic plaques, in less dense aggregates as diffuse plaques, and sometimes in the walls of small blood vessels in the brain in a process called amyloid angiopathy or congophilic angiopathy.

AD is also considered a tauopathy due to abnormal aggregation of the tau protein, a microtubule-associated protein expressed in neurons that normally acts to stabilize microtubules in the cell cytoskeleton. Like most microtubule-associated proteins, tau is normally regulated by phosphorylation; however, in AD patients, hyperphosphorylated tau accumulated as paired helical filaments that in turn aggregate into masses inside nerve cell bodies known as neurofibrillary tangles and as dystrophic neurites associated with amyloid plaques (Goedert M. et al. (2006) Tau protein, the paired helical filament and Alzheimer's disease J Alzheimers Dis 9 (3 Suppl): 195-207).

Both amyloid plaques and neurofibrillary tangles are visible by microscopy in AD brains (Tiraboschi P. et al. (2004) The importance of neuritic plaques and tangles to the development and evolution of AD Neurology 62 (11): 1984-9). At an anatomical level, AD is characterized by gross diffuse atrophy of the brain and loss of neurons, neuronal processes and synapses in the cerebral cortex and certain subcortical regions. This results in gross atrophy of the affected regions, including degeneration in the temporal lobe and parietal lobe, and parts of the frontal cortex and cingulate gyrus. Levels of the neurotransmitter acetylcholine are reduced. Levels of the neurotransmitters serotonin, norepinephrine, and somatostatin are also often reduced. Glutamate levels are usually elevated.

Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function.

The invention is also useful for the management of patients with diseases of the brain or spinal cord or diseases which affect the brain or spinal cord. Such diseases include but are not limited to: Acid Lipase Disease, Acute Disseminated Encephalomyelitis, attention deficit hyperactivity disorder, Alexander Disease, Alpers' Disease, Aneurysm, Angelman Syndrome, Arachnoiditis, Arteriovenous Malformation, Ataxia Telangiectasia, Autism, Barth Syndrome, Batten Disease, Becker's Myotonia, Behcet's Disease, Brown-Sequard Syndrome, Canavan Disease, Ceramidase Deficiency, Cerebellar Degeneration, Cerebral Beriberi, Cerebral Palsy, Cerebro-Oculo-Facio-Skeletal Syndrome, Charcot-Marie-Tooth Disease, Chiari Malformation, Cholesterol Ester Storage Disease, Choreoacanthocytosis, Chronic Inflammatory Demyelinating Polyneuropathy (CIDP), Creutzfeldt-Jakob Disease, Cushing's Syndrome, Cytomegalovirus Infection, De Morsier's Syndrome, Dementia, Subcortical Dementia, Dentate Cerebellar Ataxia, Dentatorubral Atrophy, Dermatomyositis, Developmental Dyspraxia, Devic's Syndrome, Diabetic Neuropathy, Diffuse Sclerosis, Dyslexia, Dystonias, Encephalitis, Encephalopathy, Epilepsy, Fabry's Disease, Fahr's Syndrome, Farber's Disease, Fisher Syndrome, Floppy Infant Syndrome, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Globoid Cell Leukodystrophy, Guillain-Barre Syndrome, Hallervorden-Spatz Disease, Head Injury, Herpes Zoster, Huntington's Disease, Infantile Phytanic Acid Storage Disease, Joubert Syndrome, Kearns-Sayre Syndrome, Klippel-Feil Syndrome, Kliiver-Bucy Syndrome, Korsakoff's Amnesic Syndrome, Krabbe Disease, Kugelberg-Welander Disease, Kuru, Lambert-Eaton Myasthenic Syndrome, Landau-Kleffner Syndrome, Learning Disabilities, Lesch-Nyhan Syndrome, Leukodystrophy, Levine-Critchley Syndrome, Lewy Body Dementia, Lou Gehrig's Disease, Lupus—Neurological Sequelae, Lyme Disease, Machado-Joseph Disease, Melkersson-Rosenthal Syndrome, Meningitis, Menkes Disease, Miller Fisher Syndrome, Mucolipidoses, Mucopolysaccharidoses, Multifocal Motor Neuropathy, Multi-Infarct Dementia, Multiple System Atrophy, Muscular Dystrophy, Myasthenia Gravis, Myelinoclastic Diffuse Sclerosis, Myoclonus, Narcolepsy, Neuroacanthocytosis, Neurofibromatosis, Neuroleptic Malignant Syndrome, Neurological Complications of AIDS, Neuromyelitis Optica, Neuronal Ceroid Lipofuscinosis, Neurosarcoidosis, Niemann-Pick Disease, Ohtahara Syndrome, Olivopontocerebellar Atrophy, Opsoclonus Myoclonus, O'Sullivan-McLeod Syndrome, Pain—Chronic, Pantothenate Kinase-Associated Neurodegeneration, Paraneoplastic Syndromes, Parkinson's Disease, Paroxysmal Choreoathetosis, Paroxysmal Hemicrania, Parry-Romberg, Pelizaeus-Merzbacher Disease, Pena Shokeir II Syndrome, Periventricular Leukomalacia, Phytanic Acid Storage Disease, Pick's Disease, Pituitary Tumors, Polymyositis, Pompe Disease, Postinfectious Encephalomyelitis, Primary Lateral Sclerosis, Prion Diseases, Progressive Multifocal Leukoencephalopathy, Progressive Supranuclear Palsy, Ramsay Hunt Syndromes, Rasmussen's Encephalitis, Reflex Sympathetic Dystrophy Syndrome, Refsum Disease, Rett Syndrome, Reye's Syndrome, Saint Vitus Dance, Sandhoff Disease, Schilder's Disease, Seizure Disorder, Shaken Baby Syndrome, Shingles, Shy-Drager Syndrome, Sotos Syndrome, Steele-Richardson-Olszewski Syndrome, Stiff-Person Syndrome, Striatonigral Degeneration, Sturge-Weber Syndrome, Subacute Sclerosing Panencephalitis, Sydenham Chorea, Syringohydromyelia, Tabes Dorsalis, Tardive Dyskinesia, Tay-Sachs Disease, Thyrotoxic Myopathy, Tourette Syndrome, Transmissible Spongiform Encephalopathies, Transverse Myelitis, Traumatic Brain Injury, Trigeminal Neuralgia, Tropical Spastic Paraparesis, Tuberous Sclerosis, Von Economo's Disease, Von Hippel-Lindau Disease, Von Recklinghausen's Disease, Wallenberg's Syndrome, Werdnig-Hoffman Disease, Wernicke-Korsakoff Syndrome, West Syndrome, Whipple's Disease, Williams Syndrome, Wilson's Disease, Wolman's Disease.

### EXAMPLES

### Example 1

### Method for Diagnosis and Monitoring of Multiple Sclerosis

A method is developed for multiple sclerosis and is implemented on human clinical scanners using a software package. The method involves the use of multiple methods and techniques for MRI and MRS based imaging of the brain or spinal cord. The methods can then be used in combination with each other to provide improved reproducibility, sensitivity and specificity to aid in patient management.

1. Data acquisition:

a. Implement on Philips, GE and Siemens 1.5T and 3T clinical systems

b. Anatomical MRI of whole brain

c. Diffusion tensor imaging (DTI) whole brain

d. Magnetization transfer (MT) imaging

e. H1 MRS, Short echo time TE 35 ms
i. Scout image, standardized selection of MRS slices and single voxel locations
ii. Mutli-voxel chemical shift imaging method
iii. Single voxel method bilaterally
   1. Corpus callosum
   2. Parietal lobes—above posterior horns
   3. Other standard peri-ventricular white matter (WM) area
   4. Other standard gray matter (GM) area
      2. Raw data transfer from clinical system to processing server, reading of data on server using LC model or JMRUI
      3. Data processing and analysis

a. Image unwarping/atlas-based alignment/segmentation/QC
i. Identification of standard brain regions for analysis
ii. QC: SNR, line width criteria, location of voxels and CSI slice

b. Anatomical MRI calculations
i. Calculation of whole brain area
ii. Calculation of area of specific structures and regional cortical thickness
iii. Calculation of individual and overall lesion area
iv. DTI measures of fractional anisotropy and mean diffusivity for standard locations
v. MT ratio quantification within subregional WM, GM

c. MRS calculations
i. Calculation of peaks and ratios: mI, NAA, Choline, Cr, free lipids, PC, Glutamine/Glutamate
   1. For entire brain+volume corrected metabolite ratios
   2. For standard regions with calculation of GM and WM content in region of interest
   3. For lesions+volume corrected metabolite ratios

d. Registration to prior scan (when available):
i. Calculation of change metrics for all metabolites in all regions
ii. Calculation of rate of change (time factor)
   4. Data display and reporting

a. QC information

b. Segmental volumes, regional cortical thickness, lesion load, longitudinal change, with error bounds and reference ranges

c. Anatomical MRI with heat map for
i. DTI results (fractional anisotropy and mean diffusivity)
ii. MT results (magnetization transfer ratio)
iii. MRS metabolites and ratios

d. Metabolite values for whole brain, standard regions, and lesions with reference ranges and error bounds (+volume corrected results)

e. Longitudinal DTI, MT and MRS metabolite results (numerical, heatmaps and graphical)

f. Interpretation

### Example 2

### Clinical Trial for Validation of Method for Diagnosis and Monitoring of Multiple Sclerosis: Detecting Disease Progression as Defined by New Lesion Development on Conventional Brain MRI

A clinical trial is designed and performed which validates the combination method for imaging and the associated software package. In this example, the trial demonstrates the predictive power of the combined technique for the radiological and clinical progression of relapsing and remitting multiple sclerosis and further demonstrates the utility of the combined method relative to any method used individually.

### Title A Multi-center Study To Evaluate the Predictive Value of Automated MRS/MRI Versus Conventional MRI in Detecting Disease Progression in Relapsing-Remitting Multiple Sclerosis (MS)

Study Objective To determine the predictive power of automated MRS/MRI compared to conventional MRI in detecting disease progression as defined by new lesion development on conventional brain MRI
Number of Subjects Total of 200 subjects
Accrual Period Approximately 3 months
Study Design Subjects will be assessed at 6 months and 12 months after enrollment and for new lesion development on conventional brain MRI.

### Study Procedures

This is a multi-center observational study to evaluate the predictive value of automated and standardized multi-modal MRS/MRI methods versus conventional MRI techniques in the detection of disease progression in relapsing-remitting MS (RR-MS) patients. Upon enrollment subjects will be evaluated immediately with MRS/MRI and conventional MRI. Patients will be allowed to receive standard of care (e.g. methylprednisolone or beta-interferon) throughout the study. They will be followed on a regular basis with clinical examinations and both MRS/MRI and conventional MRI techniques approximately every 8 weeks. Subjects will also be assessed with both clinical examination and MRS/MRI and conventional MRI if and when a relapse has occurred and 4 weeks after initiation of treatment for relapse.

### Study Duration

6 months for first endpoint and 12 months for second endpoint

### Primary Study Endpoint

To demonstrate the predictive power of MRS/MRI for development and progression of lesion severity on conventional MRI measures including:

a. # new or enlarging T2 hyperintense lesions

b. # of gadolinium enhancing lesions

c. Total T2 lesion volume

This endpoint will be examined both at 6 months and at 12 months.

### Secondary Study Objectives

To determine the utility of MRS/MRI for monitoring response to therapy for clinical relapses of RR-MS

### Statistical Design and Assumptions

Analysis population: The primary analysis population is subjects who have completed all 6 months of follow up with no missing visits for the 6 months analysis and 12 months for the 12 month analysis.
The initial primary analysis will be conducted after the last subject completes the 6 month visit. The total number of new or enlarging T2 hyperintense lesions on all scans to 6 months, the total number of gadolinium enhancing lesions on all scans to 6 months, and T2 lesion volume at month 6 will be calculated. The predictive power of the single baseline MRS/MRI versus conventional MRI on the above will be determined.
Further primary analysis will be conducted after the last subject completes the 12 month visit. The total number of new or enlarging T2 hyperintense lesions on all scans to 12 months, the total number of gadolinium enhancing lesions on all scans to 12 months, and T2 lesion volume at month 12 will be calculated. The predictive power of the single baseline MRS/MRI versus conventional MRI on the above will be determined.
A secondary analysis will be performed at month 12 by stratifying the patients based on severity of disease. Subjects will be stratified as follows: >20 total gadolinium enhacing lesions on screening MRI, <or=to 20 total gadolinium enhacing lesions on screening MRI. These subgroups will be analyzed in the same manner as above to determine the predictive power of the baseline MRS/MRI versus conventional MRI on the various MRI measures.
A secondary analysis will also be performed at month 12 to determine the utility of MRS/MRI in predicting clinical relapses of RR-MS. These results will be stratified based on the type of therapy that the patient received for the clinical relapse.

### Patient Population

Participants 18 to 50 years of age with a diagnosis of definite RR-MS as defined by revised McDonald criteria (Polman et. al. Ann Neurol 2005; 58:840-6) and who meet the inclusion and exclusion criteria.

### Inclusion Criteria

1. Male or female between 18 and 50 years of age.

2. Definite diagnosis of RR-MS by the revised McDonald criteria (Polman et. al. Ann Neurol 2005; 58:840-6).

3. Five new brain T2 and/or new gadolinium enhancing lesions within the last 12 months or one or more relapses within the previous 12 months.

4. EDSS 0 to 6.5 inclusive.

5. Participants who are willing to sign the study specific informed consent form.

### Exclusion Criteria

1. Primary progressive, secondary progressive or progressive relapsing MS.

2. Current or prior treatment with Tysabri

3. Clinically isolated syndrome (CIS).

4. History of systemic illness or medical condition that would limit the likelihood of completing the MRS and MRI procedures.

5. Participants with implanted pace makers, defibrillators, or metallic objects on or inside the body.

6. Major medical illnesses or psychiatric impairment that, in the investigator's opinion, will prevent completion of the protocol.

### Example 3

### Clinical Trial for Validation of Method for Diagnosis and Monitoring of Multiple Sclerosis: Detecting Disease Progression as Defined by Brain Volume and Atrophy as Measured by Conventional MRI and Clinical Measures of Disease

Another clinical trial is designed and performed which validates the combination method for imaging and the associated software package. In this example, the trial demonstrates the predictive power of the combined technique for the radiological and clinical progression of relapsing and remitting multiple sclerosis and further demonstrates the utility of the combined method relative to any method used individually.

### Title A Multi-center Study To Evaluate the Predictive Value of Automated MRS/MRI Versus Conventional MRI in Detecting Disease Progression in Relapsing-Remitting Multiple Sclerosis (MS), Extension Study

Study Objective To determine the predictive power of automated MRS/MRI compared to conventional MRI in detecting parameters of MS disease progression. These two parameters are categorized as follows:

brain volume and atrophy as measured by conventional MRI.

clinical measures of disease.

Number of Subjects Total of 200 subjects

### Study Design

This study represents an extension of protocol described in Example 2, and thus patients will be recruited from that study. After completion of the study procedures of Example 2, those same subjects will continue in this protocol and they will be assessed after an additional 12 months for: (1) whole brain volume and black holes on conventional MRI, and (2) for clinical relapses and other clinical measures.

### Study Procedures

This is a multi-center observational study to evaluate the predictive value of automated and standardized multi-modal MRS/MRI methods versus conventional MRI techniques in the detection of disease progression in relapsing-remitting MS (RR-MS) patients. Subjects will be followed on a regular basis with clinical examinations and both MRS/MRI and conventional MRI techniques approximately every 8 weeks. Subjects will also be assessed with both clinical examination and MRS/MRI and conventional MRI if and when a relapse has occurred and 4 weeks after initiation of treatment for relapse.

### Study Duration

12 months (in addition to the 12 months required for the protocol described in Example 2)

### Primary Study Endpoint

To demonstrate the predictive power of MRS/MRI for clinical relapse, neurologic disability and cognitive dysfunction

### Secondary Study Objectives

To demonstrate the predictive power of MRS/MRI for whole brain parenchymal volume loss and number and volume of T1 hyopointense lesions (T1 black holes)
To determine the utility of MRS/MRI for monitoring response to therapy for clinical relapses of RR-MS

### Statistical Design and Assumptions

Analysis population: The primary analysis population is subjects have completed all 24 months of follow up (since enrollment in protocol protocol described in Example 2) with no missing visits for 24 months.
The primary analysis will be conducted after the last subject completes the 24 month visit (after enrollment in protocol described in Example 2). The annualized clinical relapse rate, neurologic disability score and cognitive dysfunction will be determined. The predictive power of the single baseline MRS/MRI versus conventional MRI on these clinical parameters will be determined.
As a secondary analysis, these results will also be stratified based on the type of therapy that the patient received for the clinical relapse.
An additional secondary analysis will examine whole brain parenchymal volume loss and number and volume of T1 hyopointense lesions (T1 black holes) at the month 24 scan. The predictive power of the single baseline MRS/MRI versus conventional MRI on these MRI measures will be determined.

### Patient Population

Participants 18 to 50 years of age with a diagnosis of definite RR-MS as defined by revised McDonald criteria (Polman et. al. Ann Neurol 2005; 58:840-6) and who meet the inclusion and exclusion criteria.

### Inclusion Criteria

1. Participants who have completed all study procedures of protocol described in Example 2.

2. Participants who are willing to sign the study specific informed consent form.

### Exclusion Criteria

1. Participants whose MS has progressed or transformed into primary progressive, secondary progressive or progressive relapsing MS.

2. Development of systemic illness or medical condition that would limit the likelihood of completing the MRS and MRI procedures.

3. Participants with implanted pace makers, defibrillators, or metallic objects on or inside the body.

4. Major medical illnesses or psychiatric impairment that, in the investigator's opinion, will prevent completion of the protocol.

### Example 4

### Method for Diagnosis and Monitoring of Alzheimer's Disease

A method is developed for Alzheimer's disease and is implemented on human clinical scanners using a software package. The method involves the use of multiple methods and techniques for MRI and MRS based imaging of the brain or spinal cord. The methods can then be used in combination with each other to provide improved reproducibility, sensitivity and specificity to aid in patient management.

1. Data acquisition:
a. Implement on Phillips, GE and Siemens 1.5T and 3T clinical systems
b. Anatomical MRI of brain
c. H1 MRS, Short echo time TE 35 ms
   i. Scout image, standardized selection of MRS slices and single voxel locations
   ii. Mutli-voxel chemical shift imaging method for total brain and/or selected slices
   iii. Single voxel method bilaterally
      1. Posterior Cingulate Gyrus
      2. Hippocampus

2. Raw data transfer from clinical system to processing server, reading of data on server using LC model or JMRUI

3. Data processing and analysis
a. Atlas based alignment/segmentation/QC
   i. Alignment to atlas
   ii. Registration with previous scans (when available)
   iii. QC: SNR, line width criteria, location of slices and voxels
b. Anatomical MRI calculations
   i. Calculation of whole brain area
   ii. Identification of PCG and ERC of Hippocampus, calculation of structure area
   iii. Calculation of change vs. previous scans

c. MRS calculations
i. Calculation of peaks and ratios: mI, NAA, Choline, Cr, free lipids, Glutamine/Glutamate
   1. For entire brain
   2. For PCG and Hippocampus
   3. Volume corrected metabolite data for whole brain, PCG and Hippocampus
   4. Calculation of change metrics and rate of change (time factor) for all metabolites in all regions (when prior scan available)

4. Data display and reporting
a. QC information
b. Whole brain, PCG and Hippocampus areas with error bounds and reference ranges
c. Change in areas with error bounds and reference ranges
d. Anatomical MRI with heat map for metabolites and ratios
e. Metabolite values
   i. For whole brain, PCG and Hippocampus with reference ranges and error bounds
   ii. Volume corrected values for whole brain and structures
   iii. Longitudinal results (numerical, heatmaps and graphical)
f. Interpretation

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisian will recognize the interchangeability of various feature from different embodiments. Similarly, the various features and steps discussed above, as well as other known equivalents for each such feature or step, can be combined and/or exchanged by one of ordinary skill in this art to perform methods in accordance with principles described herein. Each patent, journal reference, and the like, cited herein is hereby incorporated by reference in its entirety.

Although the invention has been disclosed in the context of certain embodiments and examples, it is understood by those skilled in the art that the invention extends beyond the specifically disclosed embodiments to other alternative embodiments and/or uses and obvious modifications and equivalents thereof. Accordingly, the invention is not intended to be limited by the specific disclosure of preferred embodiments herein.

What is claimed is:

1. A method of diagnosing, monitoring, or predicting the future severity of a neurological condition in a subject comprising:
(a) performing a first magnetic resonance method on said subject to produce a first data set,
(b) performing a second magnetic resonance method on said subject to produce a second data set, wherein said second magnetic resonance method is magnetic resonance spectroscopy (MRS) which measures A/B wherein A is the amount of a first metabolite and B is the amount of a second metabolite, and
(c) analyzing said data first data set and second data set to extract mathematical features from these data sets that are then used to diagnose, monitor, or predict the future severity of a neurological condition or disease in said subject.

2. The method of claim 1, wherein said first magnetic resonance method is a magnetic resonance imaging (MRI) method selected from the group consisting of diffusion tensor imaging (DTI), anatomical resonance imaging, magnetization transfer imaging, volumetric measurements of brain, brain structures or lesions, T1 weighted MRI, T2 weighted MRI, T1 weighted MRI with contrast agents, and T2 weighted MRI with contrast agents.

3. The method of claim 1, wherein said magnetic resonance spectroscopy (MRS) is a single voxel method or a multi-voxel method.

4. The method of claim 3, wherein said multi-voxel method is chemical shift imaging.

5. The method of claim 1, wherein said magnetic resonance spectroscopy (MRS) further measures the ratio of C/D wherein C is the sum of the amounts of two or more metabolites, and D is the sum of two or more metabolites, wherein C/D does not equal one.

6. The method of claim 1, wherein said neurological condition is multiple sclerosis.

7. The method of claim 1, wherein said neurological condition is Alzheimer's Disease.

8. The method of claim 1, wherein the first and second magnetic resonance methods are used to obtain data from an anatomical region of the brain selected from the group consisting of corpus callosum, parietal lobes-posterior horns, hippocampus, and entorhinal cortex of hippocampus.

9. A method of diagnosing, monitoring, or predicting the future severity of a neurological disease or condition in a subject, comprising:
(a) performing a first magnetic resonance method on said subject to produce a first data set;
(b) performing a second magnetic resonance method on said subject to produce a second data set, wherein said second magnetic resonance method is magnetic resonance spectroscopy (MRS) which measures A/B wherein A is the amount of a first metabolite and B is the amount of a second metabolite;
(c) repeating step (a), or repeating step (b), or repeating step (a) and step (b), to generate additional data sets; and
(d) analyzing said first data set, said second data set and said additional data sets to extract mathematical features that are then used to diagnose, monitor, or predict the future severity of a neurological disease or condition in said subject.

10. The method of claim 9, wherein steps (a)-(d) are performed before and after treatment with a drug or therapy.

11. The method of claim 9, wherein step (a) and step (b) occur prior to treatment of said neurological disease or condition with a drug or therapy and step (c) occurs after treatment with said drug or therapy.

12. The method of claim 9, wherein said first magnetic resonance method is a magnetic resonance imaging (MRI) method selected from the group consisting of diffusion tensor imaging (DTI), anatomical resonance imaging, magnetization transfer imaging, volumetric measurements of brain, brain structures or lesions, T1 weighted MRI, T2 weighted MRI, T1 weighted MRI with contrast agents, and T2 weighted MRI with contrast agents.

13. The method of claim 9, wherein said magnetic resonance spectroscopy further measures the ratio of C/D wherein C is the sum of the amounts of two or more metabolites and D is the sum of two or more metabolites, wherein C/D does not equal one.

14. The method of claim 9, wherein said neurological condition is multiple sclerosis.

15. The method of claim 9, wherein said neurological condition is Alzheimer's disease.

16. The method of claim 9, wherein the first and second magnetic resonance methods are used to obtain data from an anatomical region of the brain selected from the group consisting of corpus callosum, parietal lobes-posterior horns, hippocampus, and entorhinal cortex of hippocampus.

17. The method of claim 9, wherein the metabolites are selected from the group consisting of lactate, N-acetylaspartate, glutamate, glutamine, creatine, choline, and myo-inositol.

18. The method of claim 9, wherein the metabolites comprise one or both of selected free lipids and fatty acid species.
